(19) United States
(12) Patent Application Publication
(10) Pub. No.: US 2015/0299124 A1
(43) Pub. Date: Oct. 22, 2015

(54) SYNTHESIS OF UV ABSORBING COMPOUNDS

(71) Applicant: CORAL SUNSCREEN PTY LTD, Aitkenvale (AU)

(72) Inventors: MARK YORK, VICTORIA (AU); JOHN RYAN, VICTORIA (AU)

(73) Assignee: CORAL SUNSCREEN PTY LTD, QUEENSLAND (AU)

(21) Appl. No.: 14/647,610

(22) PCT Filed: Nov. 27, 2013
(86) PCT No.: PCT/AU2013/001372

§ 371 Date: May 27, 2015

(30) Foreign Application Priority Data
Nov. 27, 2012 (AU) 2012905185
May 29, 2013 (AU) 2013901952

(51) Int. Cl. C07D 211/88; C07D 211/74; C07D 211/86; C07D 211/70

(52) U.S. Cl. CPC C07D 211/88; C07D 211/70; C07D 211/74; C07D 211/86

(57) ABSTRACT

A method of synthesis is provided to obtain a range of UV absorbing compounds. The method broadly involves (a) the reduction of a glutarimide or its reaction with a carbon nucleophile; (b) when step (a) is a reduction, exposing the product of step (a) to an acidic environment to form a cyclic amide; (c) reducing the product of step (a) or step (b) to form a corresponding enamine; and subjecting the enamine product of step (c) to an acylation.

### FIELD OF THE INVENTION

The invention relates to the field of ultraviolet light absorbing compounds. More particularly, this invention relates to a method of synthesis of ultraviolet light absorbing compounds, use thereof, and novel intermediates formed during their synthesis.

### BACKGROUND TO THE INVENTION

Any reference to background art herein is not to be construed as an admission that such art constitutes common general knowledge in Australia or elsewhere.

Ultraviolet light (UV) absorbing or screening compounds have been isolated from a range of natural sources including coral, algae and cyanobacteria. The compounds, or more typically derivatives thereof, are being investigated for possible use in a range of applications where protection from the sun's harmful UV rays is desirable. This includes their use in sun screen formulations to protect the skin of the user from damage caused by UV radiation.

Amongst the most active natural UV absorbing compounds are the mycosporine-like amino acids (MAA's) which are a family of compounds that have a peak absorption in the 310-360 nm range and absorption coefficients comparable to those of synthetic sunscreens. There has therefore been considerable focus on the isolation and characterisation of naturally occurring MAA's as well as strong interest in the generation of active derivatives and analogues thereof.

U.S. Pat. Nos. 5,352,793 and 5,637,718 describe a range of MAA analogues as UV absorbing compounds based on a cyclic enaminoketone core. Although the compounds disclosed therein are effective as UV absorbing agents the synthetic routes provided to obtain those compounds are not entirely satisfactory with a number of lengthy purification steps required and a less than optimal overall yield contributing to the considerable expense to provide any of the compounds at a commercial scale. This has limited the commercialisation of these compounds into formulations, such as sunscreens, which could otherwise have provided considerable health benefits to the public.

U.S. Pat. No. 5,352,793, for example, does not provide for variations in the substitution pattern at the 4-position of the tetrahydropyridine ring system.

It would therefore be desirable to provide for an improved method of synthesising such compounds to enable their generation in commercial quantities, for example in amounts greater than 100 g.

### OBJECT OF THE INVENTION

It is an aim of this invention to provide for a method of synthesising UV absorbing compounds which overcomes or ameliorates one or more of the disadvantages or problems described above, or which at least provides a useful alternative.

Other preferred objects of the present invention will become apparent from the following description.

### SUMMARY OF INVENTION

According to a first aspect of the invention, there is provided a method of synthesising a compound, or salt thereof, including the steps of:
(a) subjecting a glutarimide to a reduction to convert one of the carbonyl oxygen atoms into a hydroxyl group or reacting the glutarimide with a carbon nucleophile to form a cyclic amide;
(b) exposing the product of step (a), wherein that step was a reduction of the glutarimide, to an acidic environment to form a cyclic amide;
(c) reducing the cyclic amide of step (a) or step (b) to form a corresponding enamine; and
(d) subjecting the enamine product of step (c) to an acylation; to thereby form the compound or salt thereof.

Suitably, the compound is a cyclic enaminoketone compound, or salt thereof.

In one preferred embodiment the cyclic enaminoketone is a compound of formula I:

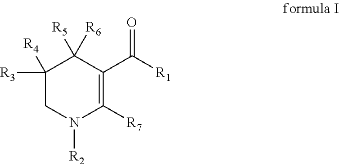

wherein, R₁ is selected from the group consisting of C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted;

R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;

R₃ and R₄ are independently selected from the group consisting of hydrogen, hydroxyl, C₁ to C₇ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;

R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and

R₇ is selected from the group consisting of hydrogen, C₁ to C₁₂ alkyl, C₂ to C₁₂ alkenyl, C₂ to C₁₂ alkynyl, aryl, C₃ to C₇ cycloalkyl, C₃ to C₇ cycloalkenyl, C₂ to C₉ alkanoyl and carbamoyl all of which groups may be substituted or unsubstituted.

Preferably, step (a) involves the reduction of a glutarimide compound of formula II or its reaction with a carbon nucleophile to give a compound of formula III or formula IV:

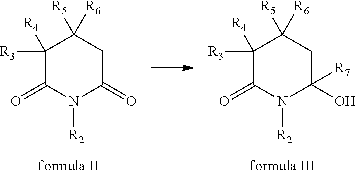

wherein, R₂, R₃, R₄, R₅, R₆ and R₇ are as previously described.

Step (b), as an entirely separate step to step (a), is optional and preferably involves exposing the compound of formula III to an acidic environment to give a cyclic amide compound of formula IV:

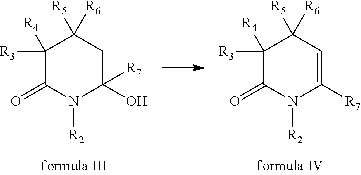

wherein, R₂, R₃, R₄, R₅, R₆ and R₇ are as previously described.

In a preferred embodiment, the product of step (a) is exposed to an acidic work up to thereby effect the conversion of step (b). In this manner steps (a) and (b), while both still performed in a step wise fashion, may be viewed as having been combined into a single reaction and work up step. That is, the present invention is not limited to step (b) being performed as a separate step after synthesis, purification and isolation of the compound of formula III are complete but rather step (b), as claimed herein, encompasses any contact between the product of step (a), for example a compound of formula III, at any time after its formation, and an acid to thereby produced a dehydrated cyclic amide analog of the product of step (a), for example a compound of formula IV, i.e. the loss of a hydroxyl group is effected.

In one embodiment, when R₇ is not hydrogen, then a reaction with a carbon nucleophile may be carried out whereby the compound of formula II proceeds directly to a compound of formula IV.

Step (c) preferably involves reducing the compound of formula IV to give an enamine compound of formula V:

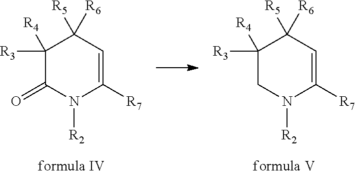

wherein, R₂, R₃, R₄, R₅, R₆ and R₇ are as previously described.

In a preferred embodiment, step (d) is then carried out to subject the enamine compound of formula V to an acylation to provide a compound of formula I:

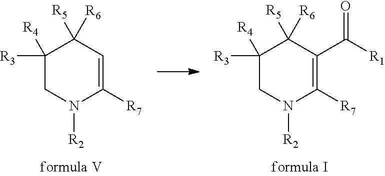

wherein, R₁, R₂, R₃, R₄, R₅, R₆ and R₇ are as previously described.

The acylation is preferably performed by reacting the enamine compound of formula V with an acyl halide or an anhydride.

Suitably, the acylation may be an alkanoylation to achieve the attachment of an R₁ group which is straight chain or branched alkyl.

The compound of formula I may be subjected to an acid treatment step to form an acidic salt of the compound of formula I.

According to a second aspect of the invention there is provided a novel compound of formula III:

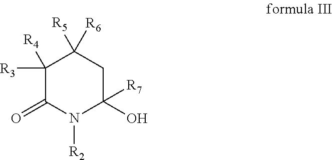

wherein, R₂, R₃, R₄, R₅, R₆ and R₇ are as previously described.

Preferably, R₇ is hydrogen.

A third aspect of the invention resides in a compound of formula I when synthesised by the method of the first aspect.

A fourth aspect of the invention resides in the use of a compound of formula I, when synthesised by the method of the first aspect, as a UV absorbing compound.

Preferably, the use of the fourth aspect is as a component of a sunscreen composition.

A fifth aspect of the invention resides in the use of a compound of the second aspect in the synthesis of a compound of formula I or in a method of synthesis of a compound of formula I comprising the transformation of a compound of formula II.

The various features and embodiments of the present invention, referred to in individual sections above apply, as appropriate, to other sections, mutatis mutandis. Consequently features specified in one section may be combined with features specified in other sections as appropriate.

Further features and advantages of the present invention will become apparent from the following detailed description.

### BRIEF DESCRIPTION OF THE DRAWINGS

In order that the invention may be readily understood and put into practical effect, preferred embodiments will now be described by way of example with reference to the accompanying figures wherein:

FIG. 1 is a synthetic scheme representing one embodiment of an improved synthesis of a compound of formula I (A855);

FIG. 2 is a synthetic scheme representing a further embodiment of an improved synthesis of a compound of formula I (A855);

FIG. 3 is a synthetic scheme similar to that in FIG. 1 representing an improved synthesis of an alternative compound of formula I (compound 319);

FIG. 4 is a ¹H NMR spectrum of a cyclic anhydride intermediate as shown in the synthetic scheme of FIG. 1;

FIG. 5 is a ¹H NMR spectrum of an open chain intermediate as shown in the synthetic scheme of FIG. 1;

FIG. 6 is a ¹H NMR spectrum of a glutarimide intermediate as shown in the synthetic scheme of FIG. 1;

FIG. 7 is a ¹H NMR spectrum of a cyclic amide intermediate as shown in the synthetic scheme of FIG. 1;

FIG. 8 is a ¹H NMR spectrum of a cyclic enamine intermediate as shown in the synthetic scheme of FIG. 1;

FIG. 9 is a ¹H NMR spectrum of the product formed in the synthetic scheme of FIG. 1 (compound A855);

FIG. 10 is a ¹³C NMR spectrum of the product formed in the synthetic scheme of FIG. 1 (compound A855);

FIG. 11 indicates the purity of the product formed in the synthetic scheme of FIG. 1 (compound A855), as shown by HPLC chromatogram; and

FIG. 12 is a UV-Vis spectrum of the product formed in the synthetic scheme of FIG. 1 (compound A855).

### DETAILED DESCRIPTION OF THE DRAWINGS

The present invention is predicated, at least in part, on the development of a greatly improved method of synthesis of certain UV absorbing compounds. The presently described method provides advantages in terms of a higher overall yield and a reduced number and/or simplification of purification steps in comparison to certain prior synthetic routes to similar compounds.

According to a first aspect of the invention, there is provided a method of synthesising a compound, or salt thereof, including the steps of:
(a) subjecting a glutarimide to a reduction to convert one of the carbonyl oxygen atoms into a hydroxyl group or reacting the glutarimide with a carbon nucleophile to form a cyclic amide;
(b) exposing the product of step (a), wherein that step was a reduction of the glutarimide, to an acidic environment to form a cyclic amide;
(c) reducing the cyclic amide of step (a) or step (b) to form a corresponding enamine; and
(d) subjecting the enamine product of step (c) to an acylation, to thereby form the compound or salt thereof.

Suitably, the method of synthesis is a method of synthesising a cyclic enaminoketone, or salt thereof.

In one preferred embodiment the cyclic enaminoketone is a compound of formula I, or salt thereof:

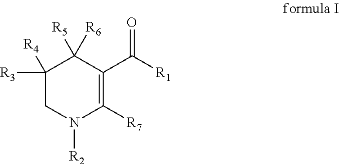

wherein, R₁ is selected from the group consisting of C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted;

R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;

R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;

R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and

R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

Suitably, R₁, R₂, R₃, R₄, R₅, R₆ and R₇ may, independently, be substituted with a substituent selected from the group consisting of hydroxyl, amino, halo, C₁ to C₆ alkoxy, C₂ to C₆ alkenoxy, C₂ to C₆ alkanoyl, C₂ to C₆ alkoxycarbonyl, carbamoyl, carbonate, carbamate, heteroaryl, and aryl.

In one preferred embodiment of the compound of formula I, R₁ is selected from the group consisting of C₁ to C₆ alkyl, C₂ to C₉ alkenyl, C₂ to C₉ alkynyl, C₂ to C₆ alkanoyl and C₂ to C₆ carbamoyl, benzyl, benzoyl and phenyl;

R₂ is selected from the group consisting of C₁ to C₉ alkyl, benzyl, phenyl, heteroaryl and C₃ to C₇ cycloalkyl;

R₃ and R₄ are independently selected from the group consisting of hydrogen, hydroxyl, C₁ to C₆ alkyl, C₁ to C₆ alkoxy and C₁ to C₆ alkanoyl; and R₅, R₆ and R₇ are independently selected from the group consisting of hydrogen, C₁ to C₆ alkyl, C₁ to C₆ alkanoyl and C₁ to C₆ alkoxy.

In a particularly preferred embodiment of the compound of formula I, R₁ is selected from the group consisting of C₁ to C₉ alkyl (which may be isoalkyl and which includes methyl, ethyl, propyl, isopropyl, n-butyl, sec-butyl, isobutyl, tert-butyl, pentyl, isoamyl and hexyl including straight chain and branched forms thereof), C₂ to C₆ alkenyl (which includes alkene equivalents of those alkyl groups recited) and C₂ to C₆ alkanoyl;

R₂ is C₁ to C₉ alkyl which includes methyl, ethyl, propyl, isopropyl, n-butyl, sec-butyl, isobutyl, tert-butyl, pentyl, isoamyl, hexyl, heptyl, octyl and nonyl including straight chain and branched forms thereof;

R₃ and R₄ are independently selected from the group consisting of hydrogen, hydroxyl, C₁ to C₆ alkyl (which may be isoalkyl and which includes methyl, ethyl, propyl, isopropyl, n-butyl, sec-butyl, isobutyl, tert-butyl, pentyl, isoamyl and hexyl including straight chain and branched forms thereof), C₁ to C₆ alkoxy and C₁ to C₆ alkanoyl; and

R₅, R₆ and R₇ are independently selected from the group consisting of hydrogen, C₁ to C₆ alkyl (which may be isoalkyl and which includes methyl, ethyl, propyl, isopropyl, n-butyl, sec-butyl, isobutyl, tert-butyl, pentyl, isoamyl and hexyl including straight chain and branched forms thereof) and C₁ to C₆ alkoxy.

In one highly preferred embodiment of the compound of formula I, R₁ is selected from the group consisting of C₁ to C₆ alkyl (which may be isoalkyl and which includes methyl, ethyl, propyl, isopropyl, n-butyl, sec-butyl, isobutyl, tert-butyl, pentyl, isoamyl and hexyl including straight chain and branched forms thereof);

R₂ is selected from the group consisting of C₁ to C₆ alkyl (which may be isoalkyl and which includes methyl, ethyl, propyl, isopropyl, n-butyl, sec-butyl, isobutyl, tert-butyl, pentyl, isoamyl and hexyl including straight chain and branched forms thereof);

R₃ and R₄ are independently selected from hydrogen or C₁ to C₆ alkyl (which may be isoalkyl and which includes methyl, ethyl, propyl, isopropyl, n-butyl, sec-butyl, isobutyl, tert-butyl, pentyl, isoamyl and hexyl including straight chain and branched forms thereof);

R₅ and R₆ are independently selected from the group consisting of hydrogen, methyl and ethyl; and

R₇ is hydrogen.

It should be understood that any one of R₁ to R₇, individually, as defined in any one of the embodiments described in the preceding paragraphs may be combined with any of the other R₁, to R₇ groups as defined in any one or more of the other embodiments described in the preceding paragraphs as if that particular combination were explicitly recited in full.

In one especially preferred embodiment, the compound of formula I is 1-(1-isobutyl-4,4-dimethyl-1,4,5,6-tetrahydropyridin-3-yl)propan-1-one or 1-(1-tert-butyl-4,4-dimethyl-1,4,5,6-tetrahydropyridin-3-yl)octan-1-one, or an acidic salt of either compound, as shown below:

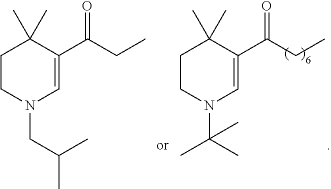

Preferably, the acidic salt is a hydrochloride salt, a sulphate salt or a sulphonate salt which is preferably an alkylated sulphonate salt.

Referring now to terminology used generically herein, the term “alkyl” means a straight-chain or branched alkyl substituent containing from, for example, 1 to about 12 carbon atoms, preferably 1 to about 9 carbon atoms, more preferably 1 to about 6 carbon atoms, even more preferably from 1 to about 4 carbon atoms, still yet more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like. The number of carbons referred to relates to the carbon backbone and carbon branching but does not include carbon atoms belonging to any substituents, for example the carbon atoms of an alkoxy substituent branching off the main carbon chain.

The term “alkenyl,” as used herein, means a linear, alkenyl substituent containing at least one carbon-carbon double bond and from, for example, 2 to 6 carbon atoms (branched alkenyls are 3 to 6 carbons atoms), preferably from 2 to 5 carbon atoms (branched alkenyls are preferably from 3 to 5 carbon atoms), more preferably from 3 to 4 carbon atoms. Examples of such substituents include vinyl, propenyl, isopropenyl, n-butenyl, sec-butenyl, isobutenyl, tert-butenyl, pentenyl, isopentenyl, hexenyl, and the like.

The term “alkynyl,” as used herein, means a linear alkynyl substituent containing at least one carbon-carbon triple bond and from, for example, 2 to 6 carbon atoms (branched alkynyls are 3 to 6 carbons atoms), preferably from 2 to 5 carbon atoms (branched alkynyls are preferably from 3 to 5 carbon atoms), more preferably from 3 to 4 carbon atoms. Examples of such substituents include ethynyl, propynyl, isopropynyl, n-butynyl, sec-butynyl, isobutynyl, tert-butynyl, pentynyl, isopentynyl, hexynyl, and the like.

Whenever a range of the number of atoms in a structure is indicated (e.g., a C₁-C₁₂, C₁-C₈, C₁-C₆, C₁-C₄, or C₂-C₁₂, C₂-C₈, C₂-C₆, C₂-C₄ alkyl, alkenyl, alkynyl, etc.), it is specifically contemplated that any sub-range or individual number of carbon atoms falling within the indicated range also can be used. Thus, for instance, the recitation of a range of 1-12 carbon atoms (e.g., C₁-C₁₂), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

The term “halo” or “halogen” or “halide” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art. It is understood that the term aryl applies to cyclic substituents that are planar and comprise 4n+2 π electrons, according to Hückel's Rule.

The term “heteroaryl” refers to an aryl group containing from one or more (particularly one to four) non-carbon atom(s) (particularly O, N or S) or a combination thereof, which heteroaryl group is optionally substituted at one or more carbon or nitrogen atom(s) with alkyl, —CF₃, phenyl, benzyl, or thienyl, or a carbon atom in the heteroaryl group together with an oxygen atom form a carbonyl group, or which heteroaryl group is optionally fused with a phenyl ring. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms.

Turning to the method of synthesis, preferably, step (a) involves the reduction of a glutarimide compound of formula II, or its reaction with a carbon nucleophile, to give a compound of formula III or formula IV, respectively. When step (a) is a reduction step then R₇ will be hydrogen. When step (a) is a reaction of a carbon nucleophile, e.g. a Grignard reagent, then a compound of Formula IV is achieved directly and the nature of R₇ will depend on the nature of the nucleophile.

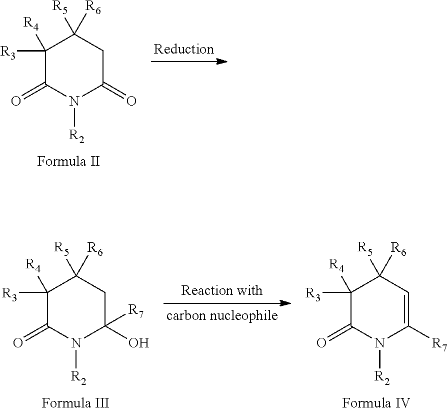

wherein, R₂, R₃, R₄, R₅, R₆ and R₇ are as previously described in any one or more of the embodiments of formula I described above.

The glutarimide compound of formula II may be a commercially available material. A number of suppliers provide glutarimides which are substituted at one or more of the R₂, R₃, R₄, R₅ and R₆ positions shown. Glutarimide itself and 3,3-dimethylglutarimide are just two such examples. In a further embodiment, as discussed further below, the present method may include steps to allow for the synthesis of a compound of formula II. In this manner any glutarimide which is required but for which a commercially available source cannot be found can be synthesised and fed into step (a).

A wide variety of reducing agents as are known in the art may be successfully employed in this reduction step. However, the optimisation of this reaction is dependent on the conditions used which were identified through considerable experimentation, as discussed below.

The reduction of the glutarimide into the corresponding hydroxyl compound of formula III is a key step in the present synthesis and the product of this reaction encompasses novel compounds which have not been reported in the literature. Initial attempts to perform this transformation using sodium borohydride (NaBH₄) as reductant gave poor results with large amounts of a ring opened compound being isolated as the major product. Performing the same NaBH₄ reduction in the presence of HCl/EtOH suppressed the ring opening process and gave the product in approximately 50% yield (when attempting the synthesis of 1-(1-isobutyl-4,4-dimethyl-1,4,5,6-tetrahydropyridin-3-yl)propan-1-one referred to hereinafter as A855) making it viable on at least a small scale. However, the reaction required a large excess of NaBH₄ (>8 mol equivalents), which would be a safety concern for larger scale synthesis, and could not be forced to completion therefore requiring the chromatographic removal of any unreacted starting material.

Attempts to perform the reduction process cleanly using sodium bis(2-methoxyethoxy) aluminumhydride (Red-Al) were also suboptimal with maximum yields of approximately 50% attainable (for the synthesis of A855) and significant amounts of starting material remaining that had to be removed chromatographically. The reaction could be forced to completion but this resulted in lower yields (approximately 30%) due to over reduction of the product to the corresponding amine.

As all glutarimide starting material could only be reduced, following these approaches, at the cost of significant loss of product to over reduction it was decided to investigate the use of lithium aluminium hydride (LiAlH₄) as a reducing agent. Although not wishing to be bound by any particular theory it was postulated that due to the lower solubility of organoaluminates derived from LiAlH₄ the desired product of the first reduction would precipitate out thereby lowering its reactivity relative to any starting material in solution. In this way, treatment of a solution of the starting glutarimide in diethyl ether with LiAlH₄ (0.52 molar equivalents) allowed approximately 80% of the desired product to be isolated.

Furthermore, incorporation of an acidic work-up into the reduction allowed conversion to the dehydrated amide product of formula IV (below) in 89% yield (for A855) with only an aqueous work-up required to isolate pure compound of formula IV. Using this process steps (a) and (b) could be combined into a single step and approximately 560 g of the starting glutarimide was transformed into approximately 440 g of the enamide product, as described in the examples for the synthesis of A855.

Replacement of the diethyl ether reaction solvent with tetrahydrofuran was investigated and found to be acceptable on a small scale. However larger scale reactions were not successful due to the tendency of the aluminates to form a single large mass in the reaction mixture making stirring and reaction quenching challenging. In contrast diethyl ether gave a well distributed powder, resulting in uniform stirring and quenching.

Thus, in one embodiment, the reduction of step (a) is carried out using an aluminium hydride based reducing agent such as lithium, sodium or potassium aluminium hydrides. The reaction is also preferably performed in an ether solvent, preferably a non-cyclic ether, most preferably diethyl ether.

In an alternative embodiment to the reduction of step (a) the compound of formula II may be exposed to a reagent generating a carbon nucleophile to thereby introduce a non-hydrogen substituent at the R₇ position. The reagent may be a Grignard or other organometallic reagent and may involve palladium catalysis. This is only a favoured approach when it is desirable to have R₇ be non-hydrogen.

Step (b) preferably involves exposing the compound of formula III to an acidic environment to give a cyclic amide compound of formula IV:

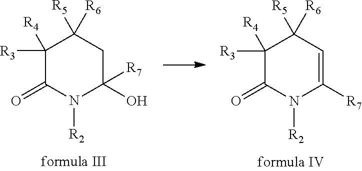

wherein, R₂, R₃, R₄, R₅, R₆ and R₇ are as previously described in any one or more of the embodiments of formula I described above.

As discussed above, in a preferred embodiment, the product of step (a) is exposed to an acidic work up to thereby effect the conversion of step (b) and give the compound of formula IV. In this manner steps (a) and (b) may be effectively combined. The work up may be a simple acidic aqueous work up.

This combined, or one-pot, reaction scheme is indicated below as an extract from the synthetic scheme towards A855. The combination of the reduction and acidification steps is indicated. As this scheme proceeded via a reduction in step (a), R₇ (not annotated) is hydrogen.

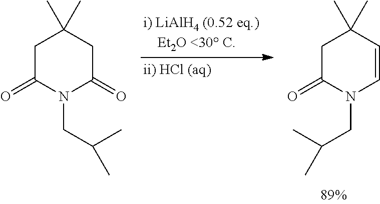

In one embodiment of the invention wherein R₇ is a non-hydrogen substituent then the alternative synthesis method discussed above employing a Grignard or like organometallic reagent may result in a transformation of the compound of formula II into a compound of formula IV without the intermediate compound of formula III being isolatable by standard techniques. Thus, step (b) is optional in that it may only be necessary when R₇ is hydrogen i.e. wherein step (a) is a reduction rather than a reaction involving a carbon nucleophile.

The carbon nucleophile generating reagent may be a reagent of formula R₈MgX wherein R₈ is C₁ to C₁₂ alkyl, preferably C₁ to C₉ alkyl, more preferably C₁ to C₆ alkyl and X is halogen. Preferably X is bromine. The reaction conditions required and range of such reagents available would be known to one of skill in the art as the Grignard reaction is a long-used and well understood reaction.

Step (c) preferably involves reducing the compound of formula IV to give an enamine compound of formula V:

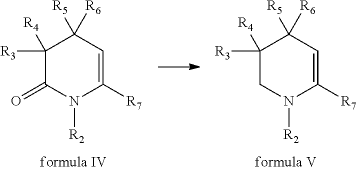

wherein, R₂, R₃, R₄, R₅, R₆ and R₇ are as previously described in any one or more of the embodiments of formula I described above.

Once again, a wide range of commercially available reducing agents may be suitable for use to achieve this transformation. However, the use of an aluminium hydride based reducing agent such as lithium, sodium or potassium aluminium hydrides has been found to be particularly useful. Lithium aluminium hydride is highly preferred. Once again, ether solvents, particularly diethyl ether are also preferred.

When this reaction was performed, as described in the examples for A855, it was found to give a 95% yield of the enamine compound. It was noted that the particular product generated en route to A855 was relatively unstable under ambient conditions and so the best approach was found to be to transfer it to the next stage of the process with the minimum of delay to avoid decomposition. The enamine could be stored under an inert atmosphere in a freezer for upwards of a week with no significant decomposition. It was also found to improve the stability of the final product if the reaction described above was performed using the addition of a relatively small amount, for example less than about 5 wt %, preferably less than about 4 wt %, more preferably less than about 3 wt %, even more preferably less than about 2 wt %, still more preferably about 1 wt %, of butylated hydroxytoluene (BHT) into the crude reaction mixture of formed compound of formula V prior to solvent removal.

Finally, step (d) is then carried out to subject the enamine compound of formula V to an acylation (alkanoylation), preferably by reaction with an acyl halide or an anhydride, to provide a compound of formula I:

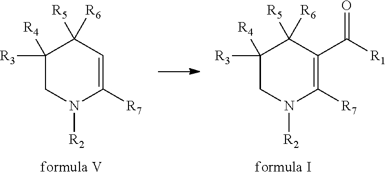

wherein, R₁, R₂, R₃, R₄, R₅, R₆ and R₇ are as previously described in any one or more of the embodiments of formula I described above.

The overall yield of the synthetic route described could be highly affected by the yield attained in step (d) which was identified as being variable due, most likely, to the quality of the compound of formula V. It was found that very little if any purification can be performed if significant decomposition is to be avoided. In the present instance, during the synthesis of A855, the reaction of step (d) followed by purification by elution from a silica pad gave approximately 85% yield of material with HPLC purity of >97%. If required, elution from a second silica pad gave the product in 75% yield with approximately 99% HPLC purity.

Temperature control was found to be critical for the optimisation of yield and product purity in this reaction. Failure to keep reaction temperature below 5° C. during the addition phase of the process resulted in a highly coloured reaction mixture and significant reductions in yield and product purity.

Thus, in a preferred embodiment, the reaction of step (d) is performed at a temperature of less than about 20° C., for example between 0° C. to 20° C., preferably less than about 15° C., for example between 0° C. to 15° C., more preferably less than about 10° C., for example between 0° C. to 10° C., and even more preferably less than about 5° C., for example between 0° C. to 5° C. Practical working temperatures include 0° C., 1° C., 2° C., 3° C., 4° C. and 5° C.

It was also found to be beneficial to the stability of the final product if a small amount of BHT, in the amounts as described above (for example about 1% weight), was added to the reaction mixture. The use of this antioxidant was found to reduce or eliminate the formation of one or more impurities which may be observed under standard conditions.

The acylation agent can be chosen from a wide range of commercially available acid halides or anhydrides. The Sigma Aldrich catalogue and other online and hard copy databases of such available chemicals provide a reference source from which the reagent which will provide the desired R₁ moiety can be chosen. For example, if R₁ is chosen as ethyl then acyl chloride could be used as the reagent. If suitable acid halide or anhydride equivalents are not available commercially to provide the desired R₁ moiety after reaction then it is common in the art to synthesise these reagents for immediate use. As such, a very wide range of reagents are available for use and so the R₁, moiety is not especially limited.

With certain of the compounds of Formula I, for example with 1-(1-isobutyl-4,4-dimethyl-1,4,5,6-tetrahydropyridin-3-yl)propan-1-one as shown below, stability issues were observed upon periods of extended storage.

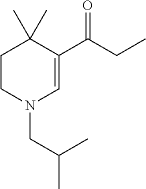 [SMILES: CCC(=O)C1=CN(CC(C)C)CCC1(C)C]

As a solution to this problem and to allow for ease of transport and long term storage it was decided to make an acid salt of the compound. The synthesis of a number of acid salts of the above compound was attempted, including those using ascorbic acid, cinnamic acid, 4-aminobenzoic acid and hydrochloric acid. Of these the conversion of the compound to the hydrochloride salt was the most successful.

Formation of the hydrochloride salt of 1-(1-isobutyl-4,4-dimethyl-1,4,5,6-tetrahydropyridin-3-yl)propan-1-one can be achieved by a number of approaches, one practical example of which is the treatment of an ethereal solution of the compound with a solution of hydrogen chloride in ether. This causes the precipitation of the hydrochloride salt of 1-(1-isobutyl-4,4-dimethyl-1,4,5,6-tetrahydropyridin-3-yl)propan-1-one from the mixture as a gum. The precipitated gum can then be heated with ethyl acetate until an off-white solid is formed. This solid can then be crushed to uniform size and heated with two further batches of ethyl acetate until the liquors fail to develop any further colouration on heating. This gives a highly stable product which does not decompose on extended storage. The hydrochloride salt can then be easily converted back to the free base by partitioning between an aqueous base, such as sodium bicarbonate, and petroleum ether. The 1-(1-isobutyl-4,4-dimethyl-1,4,5,6-tetrahydropyridin-3-yl)propan-1-one material was a pale yellow liquid which showed a HPLC purity of 100%. It is envisioned that on a larger scale the removal of impurities by the iterative washing of the suspended salt with ethyl acetate could be more efficiently realised by the use of a continuous extraction process.

The hydrochloride salt of 1-(1-isobutyl-4,4-dimethyl-1,4,5,6-tetrahydropyridin-3-yl)propan-1-one showed a UV spectra with an essentially identical λₘₐₓ to the free base (306 nm for the salt vs. 307 nm for the free base). To ensure this observation was not a result of disproportionation of the salt in the dilute methanol solution used to perform UV measurements the analysis was repeated in a non-protic solvent (THF) and gave similar results (298 nm for the salt vs. 299 nm for the free base). A sample of the salt was heated in a vacuum oven for 7 days at 50° C. to prove stability. After this time there was no discernable change in either the colour or smell of the material whereas decomposition of the free base product eventually occurs with a characteristic strong odour. Similarly there was no change in the ¹H NMR spectrum of the heated salt sample.

This observation enables the use of the more stable salt as either a long term storage medium for the compounds of Formula I or even as the UV absorbing compound itself given the showing of similar absorbance characteristics. The salt has increased solubility in water relative to the free base which, in certain formulations, may also be advantageous. If the salt of a compound of Formula I is found to be too water soluble for some sunscreen formulations then this can be reduced either by making the compound of Formula I more lipophilic or by changing the acid used to form the salt, for example to generate an alkylated sulphonic acid salt instead of hydrochloric or sulphuric acid salts.

It is a further, and important, advantage of this approach of salt formation that purification of the final compound of formula I may be achieved by salt formation and the need for chromatographic purification is completely avoided. That is, purification of the crude reaction product from the acylation step may be achieved by formation of an acidic salt, for example the hydrochloride salt, of the compound and its subsequent collection with a simple filtration and washing step. If required the salt can subsequently be readily converted to the free base, as discussed above. Otherwise the purification of the compound of formula I may, as described previously, require one or two chromatographic filtration steps. The purification by salt formation, in addition to giving cleaner final product would be expected to be considerably cheaper to implement on a large scale than the chromatographic process thereby providing a further advantage.

Thus, in one embodiment, the invention may lie in a novel acid addition salt of a compound of formula I. The salt may be the hydrochloride salt, which as described above, not only shows surprisingly effective long term stability but also maintains almost identical UV absorbing properties to the free base thereby allowing use in UV absorbing compositions, such as sunscreen compositions. Preferred acid addition salts of a compound of formula I are the hydrochloride salt of 1-(1-isobutyl-4,4-dimethyl-1,4,5,6-tetrahydropyridin-3-yl)propan-1-one or 1-(1-tert-butyl-4,4-dimethyl-1,4,5,6-tetrahydropyridin-3-yl)octan-1-one.

The method of synthesis of the first aspect as set out so far begins with the use of a glutarimide compound of formula II. As was discussed above, it may be that a glutarimide with the correct substitution pattern is not commercially available or it may simply be that, when performing a large scale synthesis, sufficient quantities cannot be reliably accessed. For this reason, in certain embodiments, the method of the first aspect may include one or more of the steps set out below leading to the synthesis of a compound of formula II.

The starting material may be a simple commercially available substituted dicarboxylic acid of formula VI which is cyclised to give the cyclic anhydride of formula VII, as set out below in step (i):

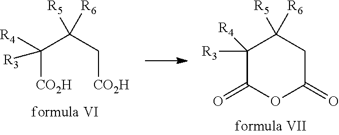

wherein, R₃, R₄, R₅ and R₆ are as previously described in any one or more of the embodiments of formula I described above.

The compound of formula VI is a relatively simple dicarboxylic acid of which many are commercially available or can be synthesised using known methods. Thus a wide range of flexibility is available in terms of the chosen R₃ to R₆ groups. The Sigma Aldrich catalogue provides access to many such dicarboxylic acids.

The reaction was initially performed using a relatively large excess of acetic anhydride (approximately 3.5 mol equivalents) which necessitated a lengthy distillation followed by a recrystallisation in order to obtain the compound of formula VII in a pure form. This gave a useful 89% yield of the product but the time and effort required for the work up meant the reaction was less than ideal for large scale work. This transformation could also be achieved using a 4 fold excess of thionyl chloride.

In an attempt to improve the efficiency of this reaction in terms of both yield and purification effort required it was decided to look to the use of continuous-flow conditions. Flow reactors for continuous flow processing are typically tubular or microfluidic chip-based systems, where reagents are introduced at different points into the tube in a continuous stream rather than in a flask or large tank (batch reactors). Because of the small dimension of the tubes and built-in automation, well defined temperature, pressure, and reaction times are achieved. This can provide advantages in practice such as ease of scale-up, high reproducibility, rapid mixing and heat transfer and inherently improved safety due to smaller reactor volumes and the containment of hazardous intermediates.

Experimentation resulted in the development of a continuous-flow system that allowed the rapid transformation of the dicarboxylic acid of formula VI (in the case of the synthesis of A855 this was 3,3-dimethylglutaric acid) to the desired anhydride of formula VII. In the synthesis of A855 this was achieved in quantitative yield and high throughput. As a much smaller excess of acetic anhydride was able to be used to effect this transformation due to the continuous-flow conditions (1.2 mol equivalents vs. 3.5 mol equivalents) the only isolation required to give the pure product was evaporation of the residual solvent. Using this process 750 g of the 3,3-dimethylglutaric acid was converted to approximately 669 g of the 3,3-dimethylglutaric anhydride. In this instance, whilst the 3,3-dimethylglutaric anhydride product (CAS#4160-82-1) is commercially available and could be a starting point for a manufacturing process, as described above, it is approximately twice as expensive as the starting acid. Additionally, analysis of commercial anhydride can sometimes show the presence of significant impurities. Thus the ability to synthesise the cyclic anhydride in high or indeed quantitative yields in such a straightforward manner is distinctly advantageous.

Therefore, in one preferred embodiment, step (i) is performed under continuous flow conditions rather than a batch synthesis.

The next stage is the reaction of the cyclic anhydride of formula VII to give the compound of formula VIII. This is shown below as step (ii).

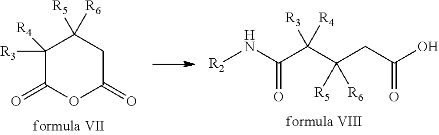

wherein, R₂, R₃, R₄, R₅ and R₆ are as previously described in any one or more of the embodiments of formula I described above.

This reaction may be performed as a solvent free process but investigations showed it was unsuitable for large scale processing. Initial small scale batch investigations into the reaction showed it to be high yielding but very exothermic, raising safety concerns about the ease with which the reaction could be controlled on a large scale. In order to avoid recourse to very dilute reaction conditions and external cooling a continuous-flow process was devised, allowing the reaction to be more readily controlled and performed safely on a large scale. This provides distinct advantages when the reaction is to be performed to provide industrial or commercial quantities of the product.

In this way a solution of the anhydride starting material in DCM, or another suitable solvent which can easily be determined based upon the solubility of the starting material, was mixed with a solution of an amine in DCM, or other suitable solvent, and the combined stream passed through a series of coils heated to 50° C. for 3 minutes. The eluent stream was then washed with dilute HCl solution to remove any excess amine and the solvent removed in-vacuo to give the product, during the synthesis of A855, in 99% yield. Using this process 664 g of 3,3-dimethylglutaric anhydride was converted into 989 g of the corresponding glutarimide product.

Thus, the reaction of step (ii) is preferably performed under continuous-flow rather than batch process conditions. The amine which is chosen for the reaction with the compound of formula VII will be chosen based upon the R₂ moiety which is desired in the final product compound of formula I. A very wide range of primary amines are commercially available and/or can be easily synthesised thereby providing a very wide choice of R₂ groups. The chemistry at this position is therefore not particularly limited.

The reaction of step (ii) may be carried out at a temperature of between 10° C. to 80° C., preferably between 20° C. to 70° C., more preferably between 30° C. to 65° C. and even more preferably between about 40° C. to about 60° C. The final temperature chosen will depend upon the reactants and, to a large extent, the solvent used in the reaction.

The final step in providing the compound of formula II is step (iii) which is shown below and which involves the cyclisation of the pentanoic acid of formula VIII to give the cyclic glutarimide of formula II.

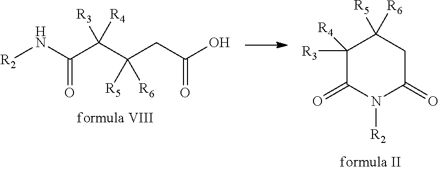

wherein, R₂, R₃, R₄, R₅ and R₆ are as previously described in any one or more of the embodiments of formula I described above.

As with step (ii), this reaction may be performed as a solvent free process but investigations showed it was unsuitable for large scale processing. Initial small scale batch experiments during the synthesis of A855 using microwave heating showed that the transformation could be effected by heating a CHCl₃ solution of the starting material to 80° C. for 10 minutes in the presence of thionyl chloride. In order to avoid the necessity of multiple small scale microwave reactions or the potentially dangerous use of a large scale sealed vessel a continuous-flow reaction was developed based upon the initial microwave experiment observations.

Thus, a solution of the starting material in CHCl₃ was mixed with a solution of thionyl chloride in CHCl₃ and the combined reagent stream passed through a series of reactor coils heated to 95° C. for 10 minutes. After an aqueous work-up the product glutarimide was obtained in 97% yield. In this way 924 g of the starting 5-(isobutylamino)-3,3-dimethyl-5-oxopentanoic acid was converted to give approximately 844 g of the product glutarimide during synthesis of A855. A range of other solvents are envisaged as being useful for this step and can be chosen based upon the solubility of the particular starting material used.

Thus, the reaction of step (iii) is preferably performed under continuous-flow rather than batch process conditions. The reactant chosen to effect the cyclisation may potentially be selected from a range of dehydrating agents. For example, various anhydrides and certain strong acids or acid halides may be suitable. A preferred dehydrating agent is thionyl chloride.

The reaction of step (iii) may be carried out at a temperature of between 10° C. to 100° C., preferably between 40° C. to 95° C., more preferably between 60° C. to 90° C. and even more preferably between about 70° C. to about 85° C. The final temperature chosen will depend upon the reactants and, to a large extent, the solvent used in the reaction.

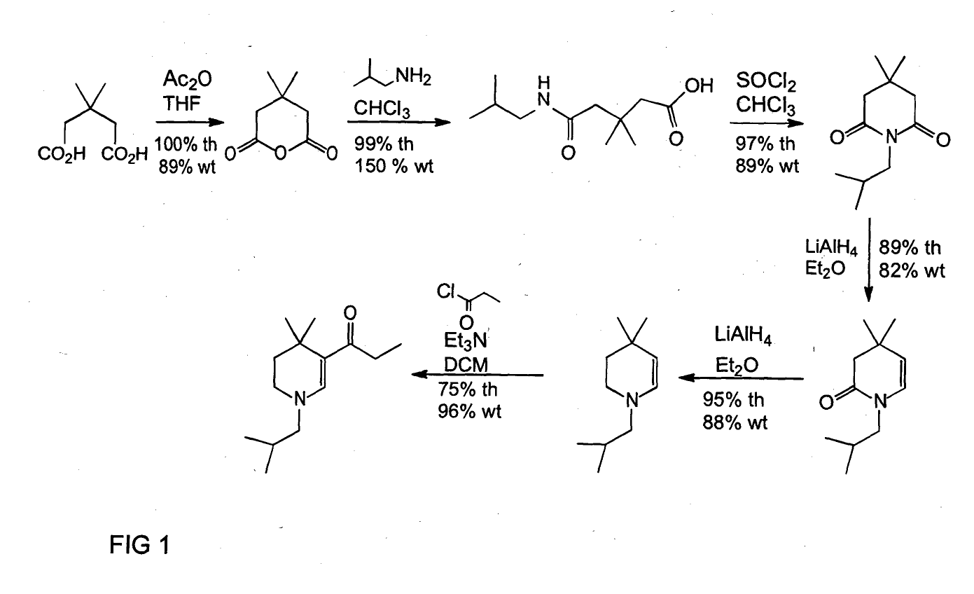

The entire synthetic scheme used to produce A855 on a 300 g scale is shown in FIG. 1. As discussed above, the scheme may simply be started after step 3 with a purchased glutarimide starting material of formula II. However, particularly for large scale synthesis of the A855 product, it is beneficial in terms of overall yield, safety and labour intensity to follow the scheme shown starting with the dicarboxylic acid of formula VI. The overall yield is 82% weight meaning that for every 100 g of A855 that is produced 122 g of the starting acid (or 108 g anhydride) would be required.

There is further scope for truncation of this process. The first three stages shown in FIG. 1 may be amenable to being combined into a single continuous-flow process. Furthermore, the two following LiAlH₄ reduction steps could be combined by adding the ether solution from the first reduction work-up to the LiAlH₄ solution for the second, thereby avoiding a solvent removal step.

In an alternative approach towards the synthesis of a compound of formula II to that outlined in steps (i) to (iii) the present method may encompass step (ia) being the N-alkylation of a glutarimide of formula IX.

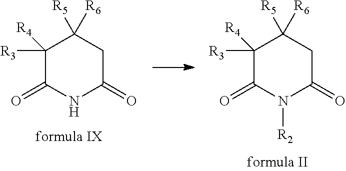

wherein, R₂, R₃, R₄, R₅ and R₆ are as previously described in any one or more of the embodiments of formula I described above.

The compound of formula IX may be available commercially or may be synthesised in a manner analogous to that outlined in steps (i) to (iii) above but without the early introduction of the R₂ group on the nitrogen via an amine.

Studies were performed on the alkylation of 3,3-dimethylglutarimide (CAS#1123-40-6) with isobutyl bromide using potassium carbonate as base in the presence of catalytic 18-crown-6. These gave an approximately 85% yield of the desired product upon heating to reflux in toluene, albeit with extended reaction times (66 hours). Thus, although not a preferred pathway, the approach of step (ia) may be useful in combination with or as a replacement for one or more of steps (i) to (iii). The N-alkylation may be limited to the use of non-tertiary organohalide reagents. If it is desirable to alkylate with a tertiary group, such as a tert-butyl group for example, then a Mitsonobu reaction may provide the desired result employing the use of the appropriate alcohol as the alkylating reagent.

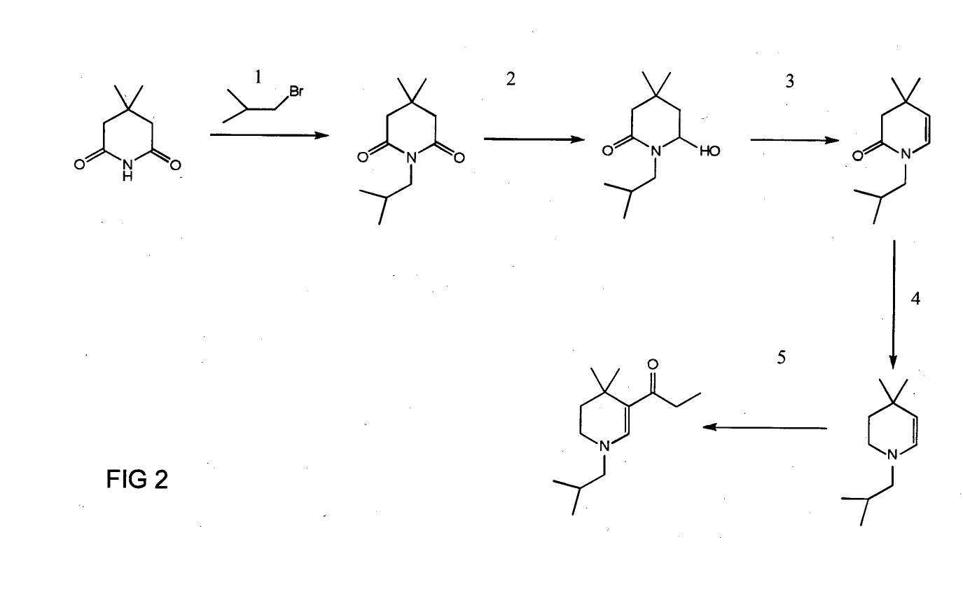

The synthetic scheme for the synthesis of A855 starting with step (ia) is shown in FIG. 2 wherein transformations 2, 3, 4 and 5 are as already described above and as indicated on FIG. 1 (steps 2 and 3 are combined i.e. ‘one-pot’ in FIG. 1).

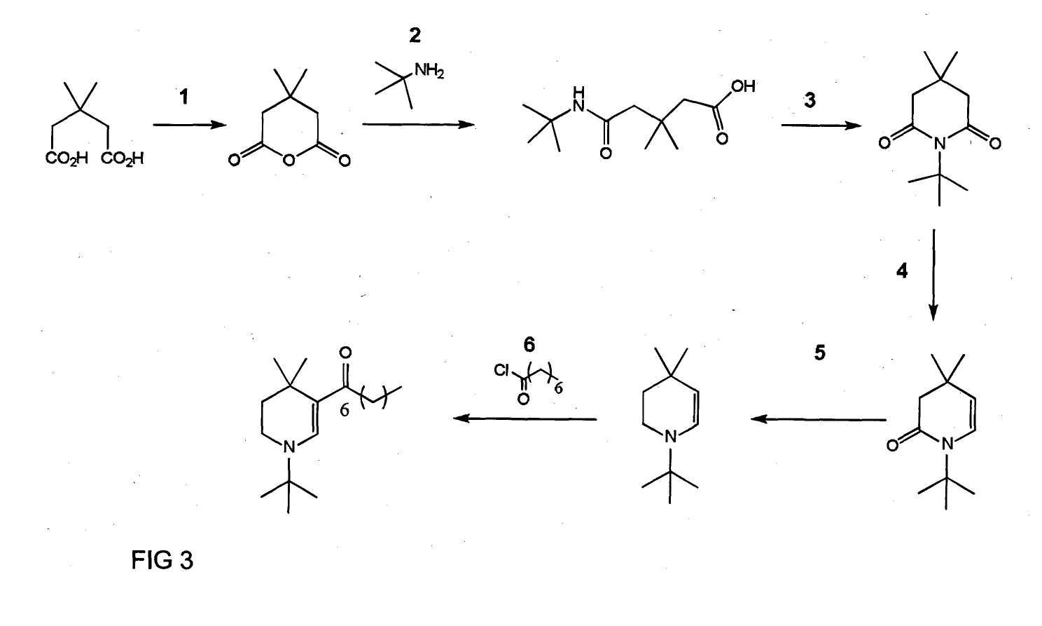

The method of synthesis of the first aspect has also been applied to the synthesis of 1-(1-tert-butyl-4,4-dimethyl-1,4,5,6-tetrahydropyridin-3-yl)octan-1-one referred to hereinafter as compound 319. This synthetic scheme is shown in FIG. 3. Again, the steps shown correspond directly to those already described above in steps (i) to (iii) and (a) to (d), and variations and alternatives thereof, with similar conditions applicable. The points of difference are in the nature of the amine of transformation 2 of FIG. 3 to provide a different R₂ moiety to that of A855 and the acid chloride used in the final transformation to provide for a longer chain R₁ group.

The general method of synthesis described herein thus has a number of advantages over prior art approaches, even those also directed to synthesis of A855. For example, U.S. Pat. No. 5,637,718 exemplifies three main synthetic routes as set out in Example 1, Example 25 and Example 26. Additionally, the patent mentions a route to final compounds starting from an alpha-dihydropyranone, but doesn't actually exemplify this route. ICI published the described route (Synth. Commun. 1993, 23, 2355).

For the route shown in Example 1 of U.S. Pat. No. 5,637,718, firstly, the starting material is not readily available and would have to be synthesised in two steps. This would involve the use of the toxic reagent mercuric acetate. Further a radical HBr addition is required which would not likely be robust on a large scale. In testing of this step difficulty was encountered with related radical HBr additions. Importantly, the overall yield of the process is less than optimal. There are potential yield losses and laborious processing because of the need to distil intermediates and purify the final product using column chromatography on silica gel. Even with column chromatography, purity of final products is not optimal.

For the route shown in Example 25 of U.S. Pat. No. 5,637,718 there are a number of problems which are also described in Synth. Commun. 1993, 23, 2355 by ICI. While a relatively short synthetic route, overall yields are moderate at best (30-40%), the conversion of the tertiary amine to the enamine suffered from moderate yields and “proved to be impractical because it required a vast excess of a potentially hazardous material, mercuric acetate (4-4.5 equivalents) and the use of hydrogen sulfide gas followed by a tedious work-up for removal of excess mercuric reagent.” Additionally, purification of intermediates by distillation is required. Further, excess acetic anhydride was used for the conversion of the anhydride into the imide. This route is also inefficient in terms of redox chemistry. The imide is fully reduced to a tertiary amine which then has to be oxidised back up to the enamine.

The route shown in Example 26 of U.S. Pat. No. 5,637,718 is an alternative approach to the production of an intermediate for Example 1. It is a lengthy synthesis and the starting material is no longer readily available. Overall yields are less than optimal due to compounding losses over the course of the lengthy synthesis. Again, distillation of intermediates is required as is chromatography of the final product. In attempting to reproduce this work in a scaled up process considerable difficulty was encountered in scaling up many of the steps. In particular, difficulties were observed with scaling up the radical HBr additions and Rosamund reduction steps.

According to a second aspect of the invention there is provided a novel compound of formula Ill:

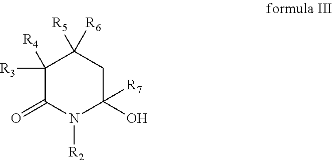

wherein, R₂, R₃, R₄, R₅, R₆ and R₇ are as previously described in any one or more of the embodiments of formula I described above.

Preferably, the compound of formula III is a compound of formula IIIa as shown below:

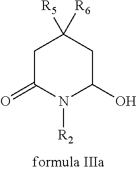

wherein, R₂, R₅ and R₆ are independently selected from C₁ to C₁₂ alkyl, C₁ to C₉ alkyl, or C₁ to C₆ alkyl which includes methyl, ethyl, propyl, isopropyl, n-butyl, sec-butyl, isobutyl, tert-butyl, pentyl, isoamyl and hexyl including straight chain and branched forms thereof.

As can be seen from the synthetic schemes shown in FIGS. 1 and 2, the compound of formula III is a key intermediate in the present synthetic approach. In one highly preferred embodiment the compound of formula III or formula IIIa is 6-hydroxy-1-isobutyl-4,4-dimethylpiperidin-2-one or 1-tert-butyl-6-hydroxy-4,4-dimethylpiperidine-2-one, as shown below.

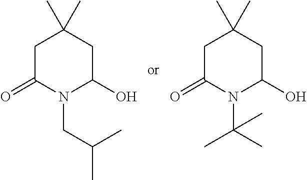

A third aspect of the invention resides in a compound of formula I when synthesised by the method of the first aspect. The method may include any of the pathways shown starting from a dicarboxylic acid, a glutarimide or cyclic anhydride.

A fourth aspect of the invention resides in the use of a compound of formula I when synthesised by the method of the first aspect as a UV absorbing compound. Such compounds are highly effective UV absorbing or screening agents and so may be useful in applications where protection from the sun's UV rays is important, such as in paint formulations or various material applications. Particularly, the compounds are effective as UV screening agents in a sunscreen formulation.

Preferably, the use of the fourth aspect is as a component of a sunscreen composition. The compound of formula I may be present in the sunscreen composition with a range of standard formulation agents including water, various emulsifiers and surfactants.

A fifth aspect of the invention resides in the use of a compound of the second aspect in the synthesis of a compound of formula I or in a method of synthesis of a compound of formula I comprising the transformation of a compound of formula III.

### Experimental

### Synthesis of A855 (1-(1-isobutyl-4,4-dimethyl-1,4,5,6-tetrahydropyridin-3-yl)propan-1-one)

The synthetic scheme for the synthesis of A855 is shown in FIG. 1 wherein the glutarimide compound is shown as progressing directly to the cyclic amide (i.e. the intermediate hydroxyl containing compound is not displayed due to the one-pot nature of the synthesis.

### Preparation of 4,4-dimethyldihydro-2H-pyran-2,6(3H)-dione (3,3-dimethylglutaric anhydride)

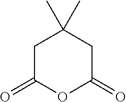

3,3-dimethylglutaric acid (375 g, 2.34 mol) was dissolved in THF to give a total solution volume of 935 mL and treated with acetic anhydride (265 mL, 2.81 mol). The solution was then pumped at a rate of 10 mL/min through a series of 4×10 mL reactor coils (PFA tubing, 1 mm i.d.) heated to 110° C. and fitted with an 8 Bar acid resistant backpressure regulator. The combined eluents were evaporated in-vacuo, toluene added (100 mL) and the mixture evaporated again to give the title compound as a colourless solid (335.0 g, 100%).

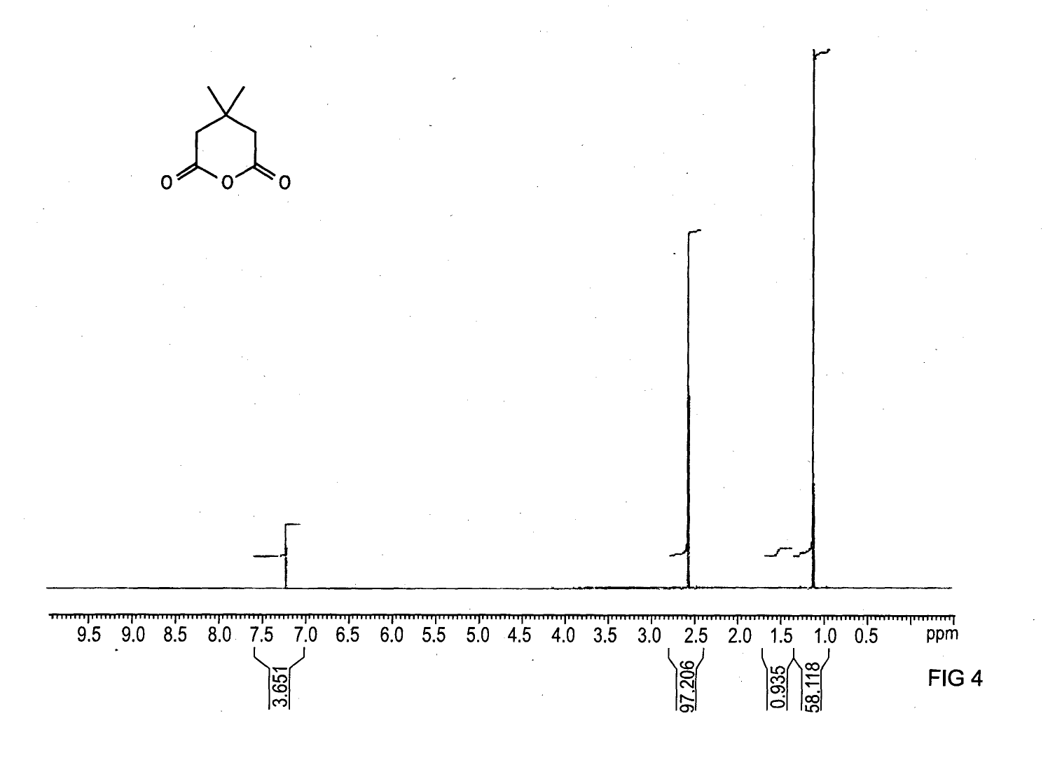

The proton NMR spectrum for this compound is shown in FIG. 4. The spectral data is as follows: δ_{H} (CDCl₃, 400 MHz) 2.62 (s, 4H), 1.17 (s, 6H).

A representative scheme of the continuous-flow conditions employed in the synthesis of 3,3-dimethylglutaric anhydride is shown below.

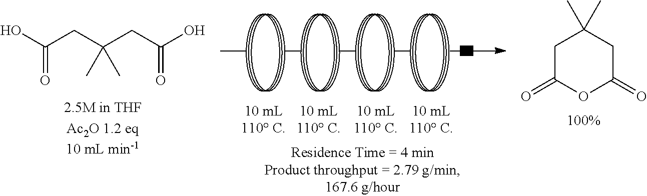

### Preparation of 5-(isobutylamino)-3,3-dimethyl-5-oxopentanoic acid

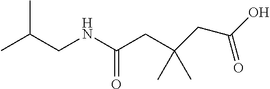

A solution of 4,4-dimethyldihydro-2H-pyran-2,6(3H)-dione (1.3 M in DCM, 1791 mL, 2.33 mol) pumped at a rate of 10 ml/min was mixed at ambient temperature with a solution of isobutylamine (5 M in DCM, 278 mL, 2.79 mol) pumped at a rate of 3.12 ml/min via a T-piece and passed through a series of 4×10 mL reactor coils (PFA tubing, 1 mm i.d.) heated to 50° C. and fitted with an 8 Bar acid resistant backpressure regulator. The combined eluents were then washed with dilute HCl solution (2 M, 500 mL), dried with magnesium sulphate and evaporated in-vacuo to a yellow oil which on standing solidified to give the title compound as a cream solid (496.6 g, 99%).

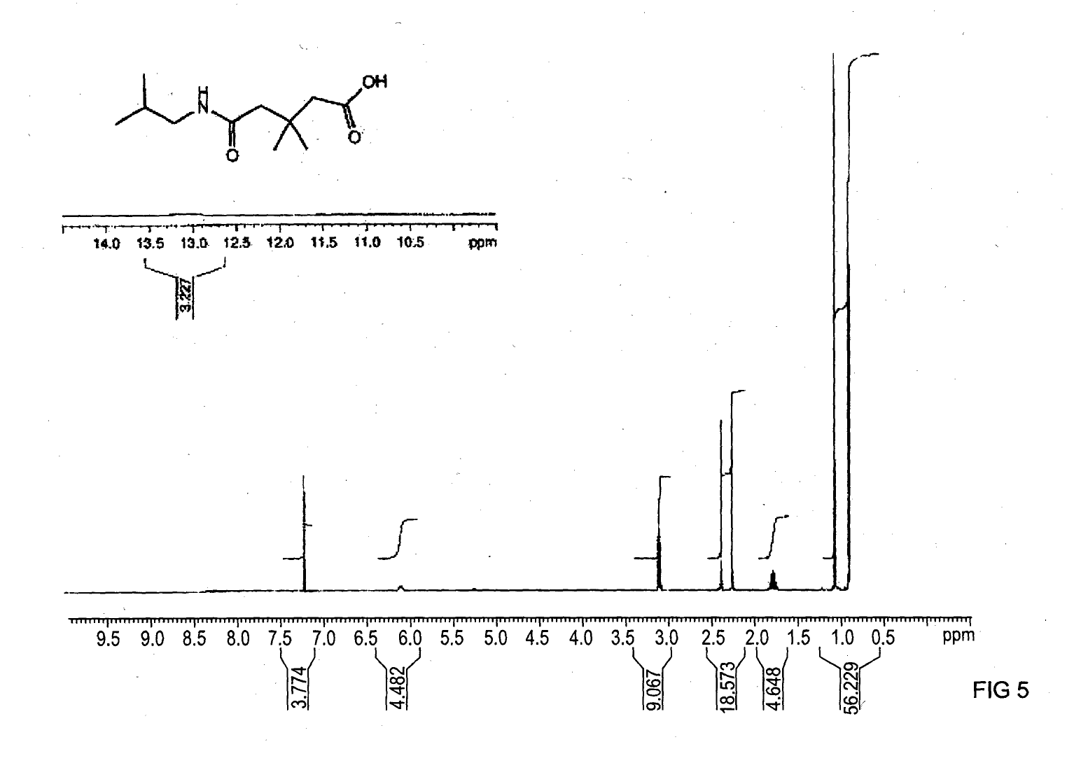

The proton NMR spectrum for this compound is shown in FIG. 5. The spectral data is as follows: δ_{H} (CDCl₃, 400 MHz) 6.17 (s, br, 1H), 3.18 (t, J 6.3, 2H), 2.45 (s, 2H), 2.33 (s, 2H), 1.90-1.80 (m, 1H), 1.14 (s, 6H), 0.97 (d, J 6.7, 6H).

A representative scheme of the continuous-flow conditions employed in the reaction of isobutylamine and 3,3-dimethylglutaric anhydride is shown below.

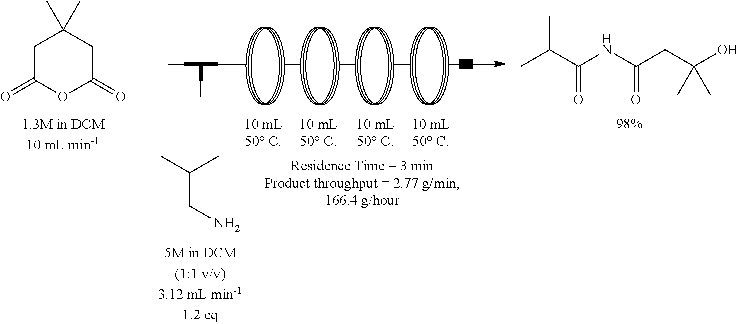

### Preparation of 1-isobutyl-4,4-dimethylpiperidine-2,6-dione (3,3-dimethyl-N-isobutylglutarimide)

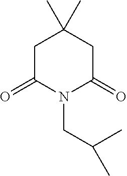

A solution of 5-(isobutylamino)-3,3-dimethyl-5-oxopentanoic acid (1.63 M in CHCl₃, 935 mL, 1.52 mol) pumped at a rate of 2.96 ml/min was mixed at ambient temperature with a solution of thionyl chloride (6.85 M in CHCl₃, 167 mL, 2.29 mol) pumped at a rate of 1.04 ml/min via a T-piece and passed through a series of 4×10 mL reactor coils (PFA tubing, 1 mm i.d.) heated to 95° C. and fitted with 2×8 Bar acid resistant backpressure regulators. The combined eluents were then evaporated in-vacuo and the residue dissolved in diethyl ether (1000 mL), washed with water (2×500 mL) and aqueous Na₂CO₃ solution (10% w/w, 500 mL). The ethereal solution was then dried with magnesium sulphate and evaporated in-vacuo to an orange oil which on standing solidified to give the title compound as a pale orange solid (291.3 g, 97%).

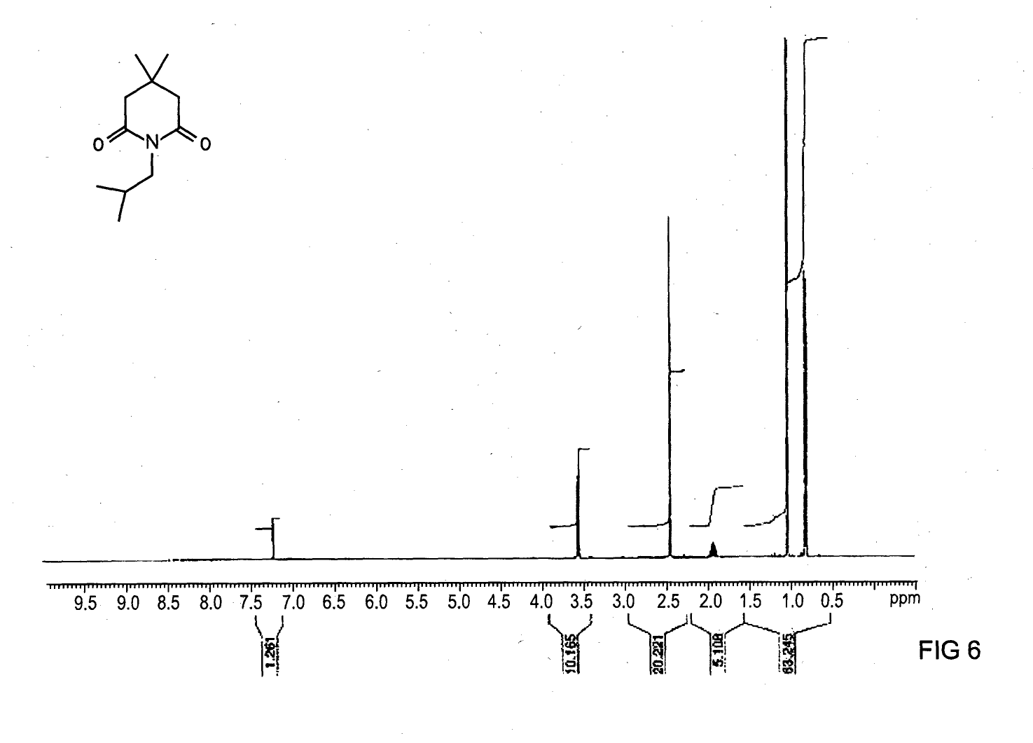

The proton NMR spectrum for this compound is shown in FIG. 6. The spectral data is as follows: δ_{H} (CDCl₃, 400 MHz) 3.63 (d, J 7.4, 2H), 2.52 (s, 4H), 2.04-1.95 (m, 1H), 1.10 (s, 6H), 0.88 (d, J 6.7, 6H).

A representative scheme of the continuous-flow conditions employed in the cyclisation of 5-(isobutylamino)-3,3-dimethyl-5-oxopentanoic acid to give the corresponding glutarimide is shown below.

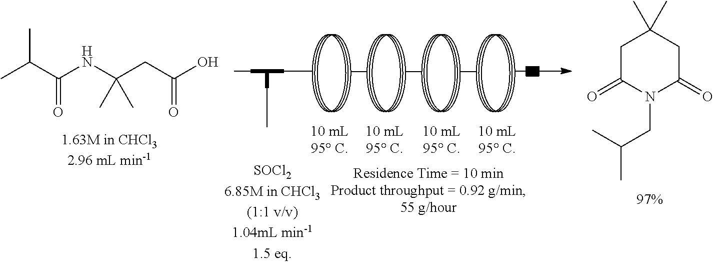

### Preparation of 6-hydroxy-1-isobutyl-4,4-dimethylpiperidin-2-one

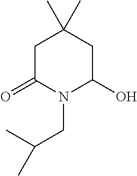

A solution of 1-isobutyl-4,4-dimethylpiperidine-2,6-dione (0.5 g, 2.53 mmol) in tetrahydrofuran (2.5 mL) was cooled on an ice bath and treated dropwise with lithium aluminium hydride (1 M in tetrahydrofuran, 2.53 mL, 2.53 mmol) at a rate sufficient to keep the temperature below 20° C. Once addition was complete a large mass of precipitate was formed which inhibited stirring. The mixture was agitated for 10 minutes and quenched by addition of sodium sulphate decahydrate (1 g, 30 mmol H₂O). The cooling batch was then removed, the mixture stirred for 10 minutes and the mixture filtered, the filter cake being washed with further portions of toluene. The combined filtrates were then evaporated in-vacuo and the residue purified by column chromatography eluting with 0-100% v/v petroleum ether/ethyl acetate. Evaporation of the product containing eluents gave 6-hydroxy-1-isobutyl-4,4-dimethylpiperidin-2-one as a pale yellow oil (0.2 g, 40%).

The proton NMR spectral data is as follows: δ_{H} (CDCl₃, 400 MHz) 4.98-4.91 (m, 1H), 3.61-3.58 (m, 1H), 3.12-3.06 (m, 1H), 2.38-1.98 (m, 5H), 1.61-1.54 (m, 1H), 1.07 (s, 3H), 1.01 (s, 3H), 0.91 (d, 3H), 0.85 (d, 3H).

### Preparation of 1-isobutyl-4,4-dimethyl-3,4-dihydropyridin-2(1H)-one

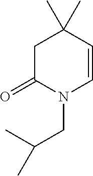

A solution of 1-isobutyl-4,4-dimethylpiperidine-2,6-dione (118 g, 544 mmol) in diethyl ether (590 mL) was cooled on an ice bath and treated dropwise with lithium aluminium hydride (1 M in diethyl ether, 283 mL, 283 mmol) at a rate sufficient to keep the temperature below 30° C. Once addition was complete (approximately 20 minutes) the mixture was stirred for 10 minutes and quenched by addition of dilute HCl solution (2 M, 40 mL) followed by further HCl solution (4 M, 450 mL) until a clear biphasic solution was obtained. The cooling batch was then removed and the mixture stirred for 25 minutes and the aqueous phase discarded. The organic phase was then dried with magnesium sulphate and evaporated in-vacuo to give the title compound as a pale orange oil (87.6 g, 89%).

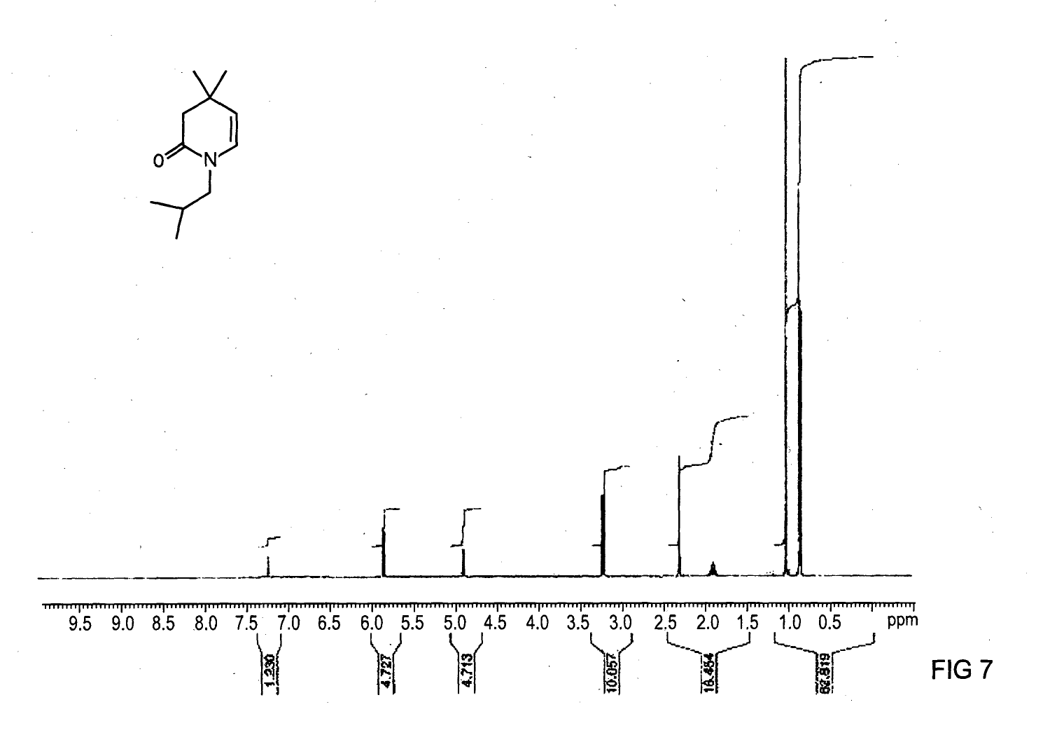

The proton NMR spectrum for this compound is shown in FIG. 7. The spectral data is as follows: δ_{H} (CDCl₃, 400 MHz) 5.90 (d, J 7.8, 1H), 4.95 (d, J 7.8, 1 H), 3.28 (d, J 7.4, 2H), 2.36 (s, 2H), 2.04-1.91 (m, 1H), 1.08 (s, 6H), 0.91 (d, J 6.7, 6H).

### Preparation of 1-isobutyl-4,4-dimethyl-1,2,3,4-tetrahydropyridine

Lithium aluminium hydride pellets (11.52 g, 303 mmol) were added to diethyl ether (250 mL) and stirred at ambient temperature for 20 minutes before treating dropwise with 1-isobutyl-4,4-dimethyl-3,4-dihydropyridin-2(1H)-one (55 g, 303 mmol) in diethyl ether (250 mL) at a rate sufficient to maintain a gentle reflux. Once addition was complete (approximately 20 min) the mixture was heated to reflux for a further 1 hour then quenched by portionwise addition of sodium sulphate decahydrate (25.9 g, 804 mmol). The resulting suspension was then stirred for 20 minutes, treated with anhydrous sodium sulphate (10 g) and stirred for a further 10 minutes before being filtered into a flask containing 1% weight of BHT (with 1% weight calculated assuming 100% yield). The filter pad was washed with diethyl ether (2×100 mL) and the combined filtrates dried with sodium sulphate and evaporated in-vacuo to give the title compound as a pale yellow liquid (48.2 g, 95%).

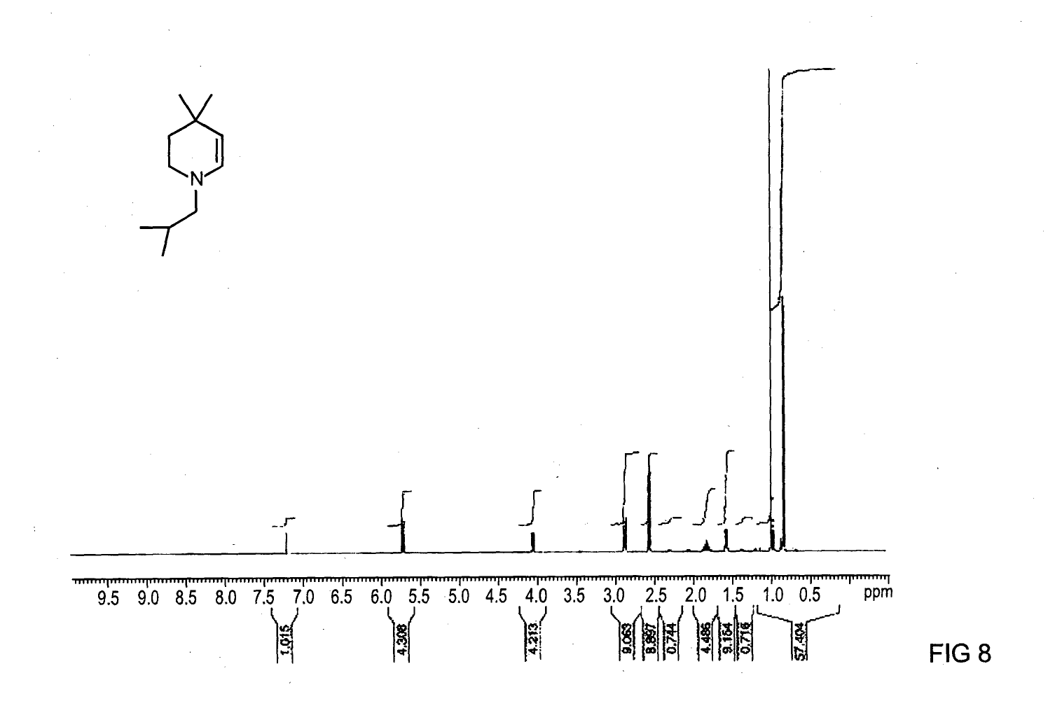

The proton NMR spectrum for this compound is shown in FIG. 8. The spectral data is as follows: δ_{H} (CDCl₃, 400 MHz) 5.78 (d, J 7.9, 1H), 4.10 (d, J 7.9, 1H), 2.92 (t, J 5.7, 2H), 2.61 (d, J 7.3, 2H), 1.90-1.83 (m, 1H), 1.60 (t, J 5.5, 2H), 1.02 (s, 6H), 0.88 (d, J 6.6, 6H).

### Preparation of 1-(1-isobutyl-4,4-dimethyl-1,4,5,6-tetrahydropyridin-3-yl)propan-1-one

A solution of 1-isobutyl-4,4-dimethyl-1,2,3,4-tetrahydropyridine (43.5 g, 260 mmol) and triethylamine (34.5 mL, 248 mmol) in DCM (250 mL) was treated with BHT (1% wt with respect to the starting enamine), cooled on an ice bath and treated dropwise with a solution of propionyl chloride (21.62 mL, 248 mmol) in DCM (150 mL) at a rate sufficient to keep the temperature of the solution below 5° C. 1% weight of BHT may be added to the reaction mixture to reduce the formation of certain impurities. Once addition was complete (approximately 25 minutes) the mixture was stirred for a further 45 minutes before quenching with water (300 mL) and stirring vigorously for a further 10 minutes. The organic phase was then separated, washed with sodium carbonate solution (10% w/w, 250 mL) and dried with sodium sulphate. Evaporation in-vacuo gave the crude material as an orange oil (57.4 g) which was purified by elution from a silica pad (approximately 6 weights silica) with 0-5% diethyl ether: DCM (1.5 L). Evaporation of the eluents gave a light orange oil (48.4 g, 88%) of approximately 97% purity. The crude material was then eluted from a second silica pad (approximately 6 weights silica, conditioned with petroleum ether, petroleum ether eluents discarded) with 30% diethyl ether: petroleum ether (1.5 L). Evaporation of the eluents gave the title compound as a yellow liquid (41.66 g, 75%) of greater than 98% purity.

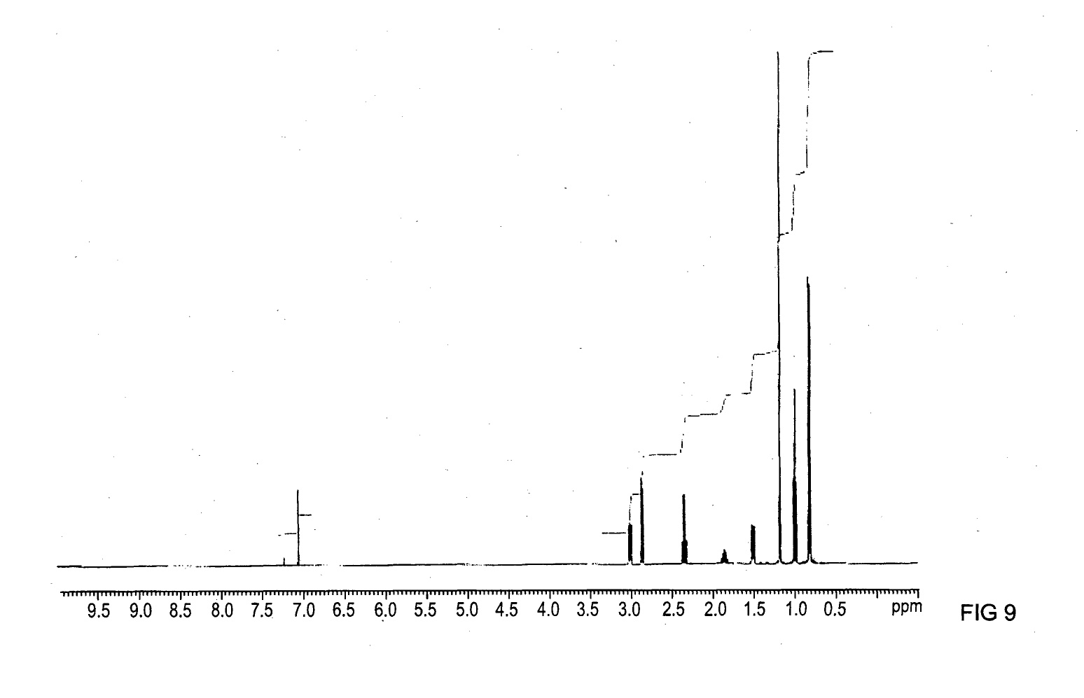

The proton NMR spectrum for this compound is shown in FIG. 9. The spectral data is as follows: δ_{H} (CDCl₃, 400 MHz) 7.15 (s, 1H), 3.12 (t, J 5.8, 2H), 2.96 (d, J 7.4, 2H), 2.46 (q, J 7.5, 2H), 2.00-1.91 (m, 1H), 1.62 (t, J 5.9, 2H), 1.29 (s, 6H), 1.10 (t, J 7.5, 3H), 0.92 (d, J 6.7, 6H).

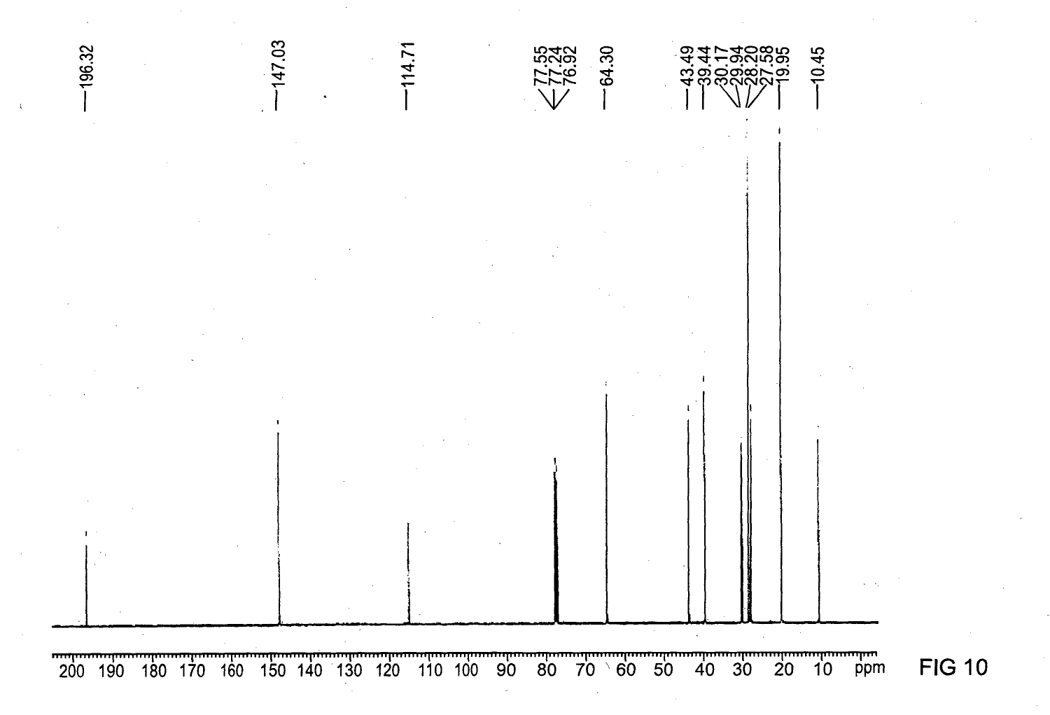

The carbon NMR spectrum for this compound is shown in FIG. 10. The spectral data is as follows: δ_{C} (CDCl₃, 100 MHz) 196.3, 147.9, 114.7, 64.3, 43.5, 39.4, 30.2, 29.9, 28.2, 27.6, 20.0, 10.5.

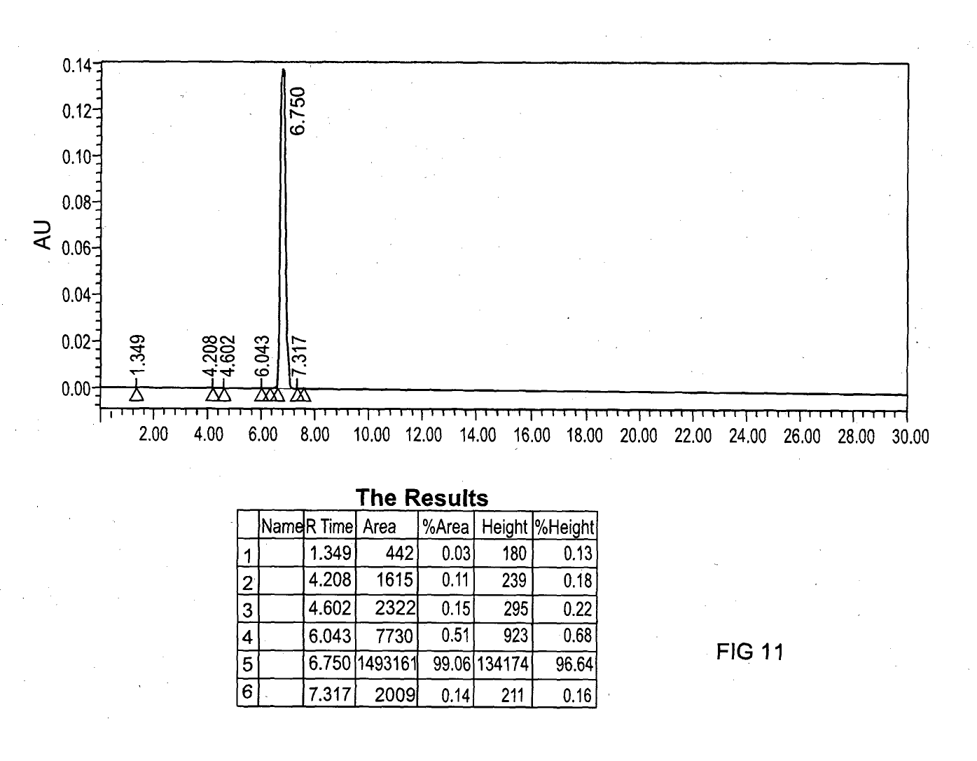

FIG. 11 indicates the purity of the product obtained as seen by HPLC chromatogram.

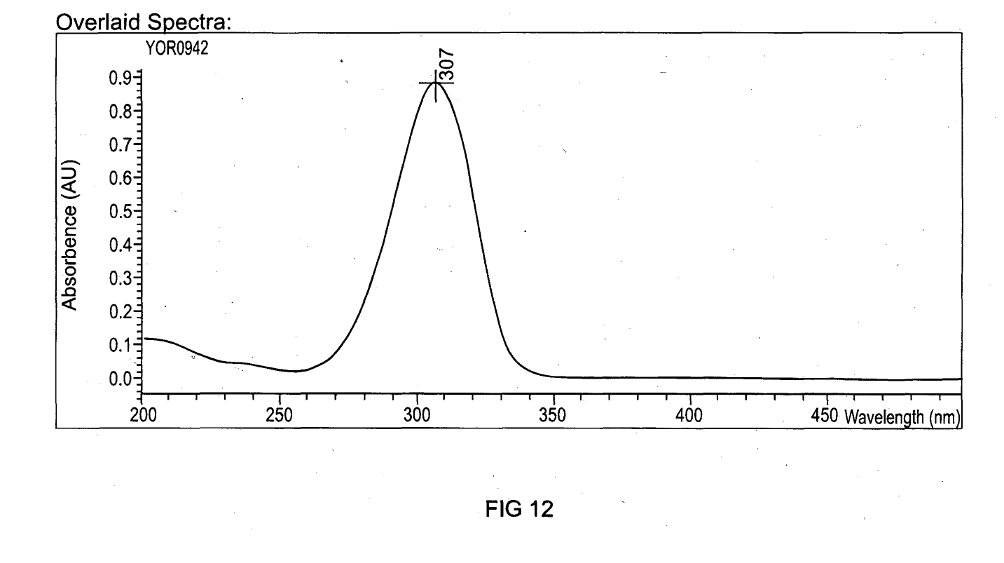

FIG. 12 is a UV-Vis spectrum of the product with the key peak being: UV λₘₐₓ 307 nm.

### Preparation of the hydrochloride salt of 1-(1-isobutyl-4,4-dimethyl-1,4,5,6-tetrahydropyridin-3-yl)propan-1-one

A portion of the 1-(1-isobutyl-4,4-dimethyl-1,4,5,6-tetrahydropyridin-3-yl)propan-1-one product (2 g) was dissolved in diethyl ether (15 mL) and treated drop wise with a 2 M solution of hydrogen chloride in diethyl ether (6.72 mL, 13.43 mmol) whilst stirring. The mixture was then evaporated in-vacuo and the resulting yellow gum treated with ethyl acetate (20 mL) and heated to reflux until a yellow solid had formed. The mixture was then cooled to room temperature, the solid crushed to uniform size and filtered. The solid was then resuspended in ethyl acetate (30 mL) and heated to reflux with stirring for 30 minutes. The yellow liquors were then removed by filtration and the solid residue resuspended in ethyl acetate (30 mL) and heated to reflux for 30 minutes. The almost colourless liquors were then removed by filtration and the very slightly off-white solid oven dried at 50° C. to give the hydrochloride salt in 79% recovery based upon starting material. δ_{H} (DMSO-d₆, 400 MHz) 8.15 (s, 1H), 3.41-3.34 (m, 4H), 2.64 (q, J 7.5, 2H), 2.08-1.99 (m, 1H), 1.62 (t, J 5.7, 2H), 1.20 (s, 6H), 1.07 (t, J 7.5, 3H), 0.86 (d, J 6.6, 6H).

UV λₘₐₓ 306 nm.

A portion of this hydrochloride salt (3.4 g) was then suspended between petroleum ether (50 mL) and sodium carbonate solution (10% w/w, 75 mL) and shaken until the solid was completely dissolved. The organic phase was then dried with magnesium sulfate and evaporated in-vacuo to give the product as a pale yellow oil with no detectable odour and an HPLC purity of 100% (2.7 g, 73% recovery) Spectral data were identical to those reported above.

The present invention thus provides for a new method of synthesising compounds of formula I, and their acid addition salts, which are useful as sunscreen agents, particularly in sunscreen compositions for human use. The method disclosed herein provides distinct advantages over those of the prior art. The advantages are particularly realised and the benefit maximised when it is required to synthesis multi-gram quantities of the target compound. For example, in the synthesis of greater than 50 g, preferably greater than 100 g, quantities the present method provides excellent overall yield with a relatively low requirement for extensive purification techniques, such as column chromatography, while maintaining a good safety profile. Steps (i) to (iii) also provide a very useful option when the cyclic anhydride or glutarimide starting materials are not available with the desired substitutions or cost or availability is limiting.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless defined otherwise, all technical and scientific terms used herein have the same meaning as would be commonly understood by those of ordinary skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. It is expected that skilled artisans will employ such variations as appropriate and it is considered within the scope and spirit of the present invention for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of synthesising a compound, or salt thereof, including the steps of:
(a) subjecting a glutarimide to a reduction to convert one of the carbonyl oxygen atoms into a hydroxyl group or reacting the glutarimide with a carbon nucleophile to form a cyclic amide;
(b) exposing the product of step (a), wherein that step was a reduction of the glutarimide, to an acidic environment to form a cyclic amide;
(c) reducing the cyclic amide of step (a) or step (b) to form a corresponding enamine; and
(d) subjecting the enamine product of step (c) to an acylation,
to thereby form the compound or salt thereof.

2. The method of claim 1 wherein the compound is a cyclic enaminoketone compound, or salt thereof.

3. The method of claim 1 wherein the compound is a compound of formula I, or a salt thereof: 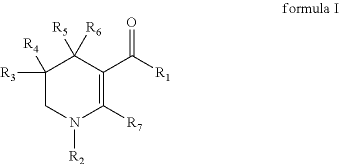
wherein, R₁ is selected from the group consisting of C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted;
R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and
R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

4. The method of claim 3 wherein R₁ is selected from the group consisting of C₁ to C₉ alkyl, C₂ to C₉ alkenyl, C₂ to C₉ alkynyl, C₂ to C₆ alkanoyl and C₂ to C₆ carbamoyl, benzyl, benzoyl and phenyl.

5. (canceled)

6. The method of claim 3 wherein R₂ is selected from the group consisting of C₁ to C₉ alkyl, benzyl, phenyl, heteroaryl and C₃ to C₇ cycloalkyl.

7. (canceled)

8. The method of claim 3 wherein R₃ and R₄ are independently selected from the group consisting of hydrogen, hydroxyl, C₁ to C₆ alkyl, C₁ to C₆ alkoxy and C₁ to C₆ alkanoyl.

9. (canceled)

10. The method of claim 3 wherein R₅, R₆ and R₇ are independently selected from the group consisting of hydrogen, C₁ to C₆ alkyl, C₁ to C₆ alkanoyl and C₁ to C₆ alkoxy.

11. (canceled)

12. The method of claim 3 wherein the compound of formula I is selected from the group consisting of 1-(1-isobutyl-4,4-dimethyl-1,4,5,6-tetrahydropyridin-3-yl)propan-1-one, 1-(1-tert-butyl-4,4-dimethyl-1,4,5,6-tetrahydropyridin-3-yl)octan-1-one and acidic salts thereof.

13. The method of claim 3 further comprising the step of forming a hydrochloride acid addition salt of the compound of formula I.

14. (canceled)

15. The method of claim 1 wherein step (a) involves reducing a compound of formula II to give a compound of formula III or reacting a compound of formula II with a carbon nucleophile to give a compound of formula IV: 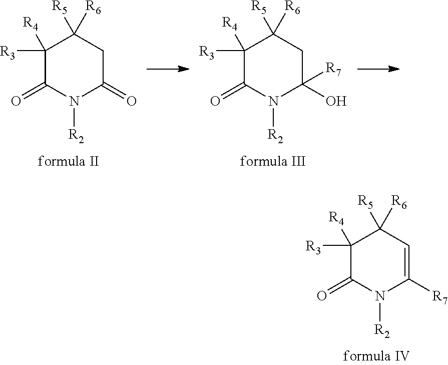
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and
R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

16. The method of claim 15 wherein, when step (a) is a reduction, step (b) involves exposing the compound of formula III to an acidic environment to form give a compound of formula IV: 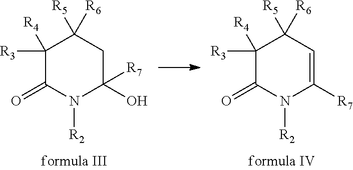
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and
R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

17. The method of claim 16 wherein step (c) involves reducing the compound of formula IV, from step (a) or step (b), to give a compound of formula V: 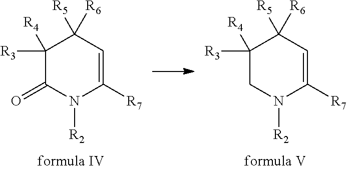
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and
R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

18. The method of claim 17 wherein step (d) involves subjecting the compound of formula V to an acylation to provide a compound of formula I, or a salt thereof: 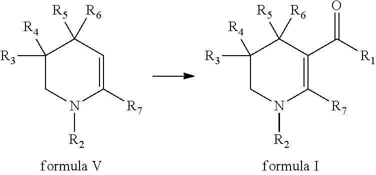
wherein, R₁ is selected from the group consisting of C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted;
R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and
R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

19. (canceled)

20. The method of claim 16 wherein the exposure to an acidic environment in step (b) occurs during work up of the reaction mixture of the reduction reaction of step (a).

21.-24. (canceled)

25. The method of claim 18 wherein the acylation in step (d) is performed at a temperature below 20° C.

26.-29. (canceled)

30. The method of claim 1 wherein the glutarimide starting material of step (a) is synthesised from a dicarboxylic acid via a cyclic anhydride and wherein the reaction of the dicarboxylic acid to give the cyclic anhydride is a reaction of a dicarboxylic acid compound of formula VI to give a cyclic anhydride of formula VII: 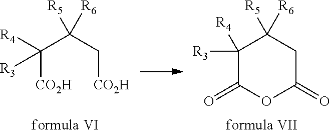
wherein, R₃, R₄, R₅ and R₆, independently, are as defined in any one of the previous claims
wherein, R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring; and
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring.

31. The method of claim 30 wherein the cyclic anhydride of formula VII is subsequently reacted with an amine to give a compound of formula VIII: 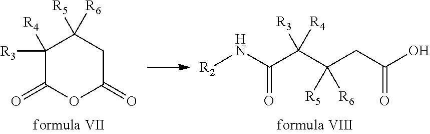
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring; and
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring.

32. The method of claim 31 wherein the compound of formula VIII is cyclised to provide the compound of formula II: 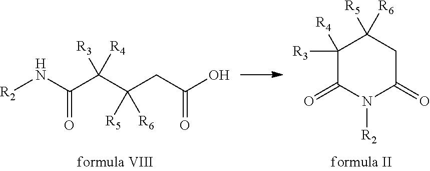
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring; and
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring.

33. The method of claim 15 wherein the compound of formula II is formed by reaction at the ring nitrogen of a compound of formula IX: 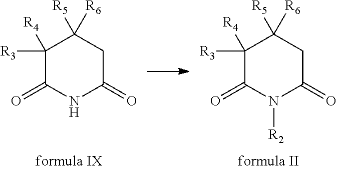
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring; and
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring.

34. (canceled)

35. A compound of formula III: 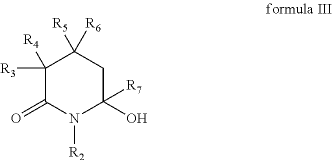
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted;
R₃ and R₄ are independently selected from the group consisting of hydrogen, hydroxyl, C₁ to C₆ alkyl, C₁ to C₆ alkoxy and C₁ to C₆ alkanoyl, each of which groups may be substituted or unsubstituted, and wherein R₃ and R₄ may together form a substituted or unsubstituted five or six membered ring;
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring; and
R₇ is selected from the group consisting of hydrogen, C₁ to C₁₂ alkyl, C₂ to C₁₂ alkenyl, C₂ to C₁₂ alkynyl, aryl, heteroaryl, C₃ to C₇ cycloalkyl, C₃ to C₇ cycloalkenyl, C₂ to C₉ alkanoyl and carbamoyl all of which groups may be substituted or unsubstituted.

36. The compound of claim 35 wherein the compound is a compound of formula IIIa: 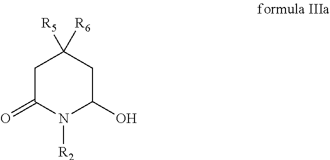
wherein, R₂ is selected from the group consisting of C₁ to C₁₂ alkyl, aryl, heteroaryl, C₃ to C₇ cycloalkyl and C₃ to C₇ cycloalkenyl, all of which groups may be substituted or unsubstituted; and
R₅ and R₆ are independently selected from the group consisting of hydrogen, C₁ to C₆ alkyl and C₁ to C₆ alkoxy, each of which groups may be substituted or unsubstituted, and wherein R₅ and R₆ may together form a substituted or unsubstituted five or six membered ring.

37.-39. (canceled)
